(19) United States
(12) Patent Application Publication
(10) Pub. No.: US 2015/0138552 A1
(43) Pub. Date: May 21, 2015

(54) APPARATUS AND METHOD FOR MEASURING PHYSIOLOGICALLY ACTIVE SUBSTANCE OF BIOLOGICAL ORIGIN

(71) Applicant: KOWA COMPANY, LTD., Aichi (JP)

(72) Inventor: Taisuke Hirono, Shizuoka (JP)

(21) Appl. No.: 14/402,948

(22) PCT Filed: Nov. 15, 2012
(86) PCT No.: PCT/JP2012/080253

§ 371 Date: Nov. 21, 2014

Related U.S. Application Data
(60) Provisional application No. 61/651,997, filed on May 25, 2012.

(51) Int. Cl. G01N 21/49; G01N 33/487

(52) U.S. Cl. CPC G01N 21/49; G01N 33/487
USPC 356/337

(57) ABSTRACT

With regard to the detection of a physiological active substance of biological origin and the measurement of its concentration in a sample, the invention provides a technique for moving gel particles that are produced in the sample without using a mechanical stirring member, and allows a highly accurate detection of the physiological active substance of biological origin and measurement of its concentration with a simple arrangement. By partial heating/cooling of a sample cell, thermal convection is generated within a mixture liquid in the sample cell, and as a result the gel particles that are produced in the mixture liquid are moved. In addition, based on the intensity of forward scattered light, the rate of change in the number of gel particles is measured.

### TECHNICAL FIELD

The present invention relates to a measuring method and a measurement apparatus for detecting or measuring the concentration of a physiologically active substance of biological origin in a sample based on detection of aggregation or gelation of proteins derived from a reaction between an AL and a physiologically active substance of biological origin that are contained in a sample mixture liquid containing an AL reagent and a predetermined physiologically active substance of biological origin.

### BACKGROUND ART

Endotoxin is a lipopolysaccharide present in a cell wall of a Gram-negative bacterium and is the most typical pyrogen. If a transfusion, a pharmaceutical for injection, dialysis liquid, blood, or the like contaminated with the endotoxin enters into a human body, the endotoxin may induce severe side effects such as fever and shock. Therefore, it is required to manage the above-mentioned pharmaceuticals so that they are not contaminated with endotoxin.

Meanwhile, a serine protease, which is activated by endotoxin, is present in hemocyte extract (herein below, also referred to as “AL: Amoebocyte Lysate”) of limulus. When AL reacts with endotoxin, according to an enzyme cascade caused by serine protease that is activated depending on the amount of endotoxin, the coagulogen present in the AL is hydrolyzed to coagullin and associated with each other to yield insoluble gel. By taking advantage of this property of AL, it is possible to detect endotoxin with a high sensitivity.

Meanwhile, β-D-glucan is a polysaccharide that constitutes a cell membrane characteristic of a fungus. Measurement of β-D-glucan is effective, for example, for broad range screening of infectious diseases due to a variety of fungi including not only fungi that are frequently observed in general clinical practices, such as Candida, Aspergillus, or Cryptococcus, but also rare fungi.

Also in this case, it is possible to detect β-D-glucan with high sensitivity by using the property of the amoebocyte lysate component AL of limulus to coagulate (coagulate to form a gel) by β-D-glucan.

As a specific method for detecting the presence of or measuring the concentration of a physiologically active substance of biological origin (hereinafter, also referred to as a “predetermined physiologically active substance”) such as endotoxin and β-D-glucan using an amoebocyte lysate component AL of limulus, there is a semi-quantitative gelation and inversion method in which a mixture liquid of a sample for detection or concentration measurement of a predetermined physiologically active substance (hereinafter, also simply referred to as a “measurement of predetermined physiologically active substance”) and AL is left standing, inverting the vessel after a certain period of time, determining an occurrence of gelation based on down flow of the mixture liquid, and examining whether or not the predetermined physiologically active substance is contained at certain concentration or more in a sample. There is also a turbidimetric method in which turbidity of a sample due to the gel formation by a reaction between AL and the predetermined physiologically active substance is measured over time and analyzed, and a colorimetric method in which change in color of a sample is measured by using a synthetic substrate which is hydrolyzed by enzyme cascade.

In the case of measuring the predetermined physiologically active substance with the above turbidimetric method, a mixture liquid of the measurement sample and the AL is generated in a dry-heat sterilized measurement glass cell. After that, gelation of the mixture liquid is illuminated with light from outside and transmittance change of the mixture liquid caused by gelation is optically determined. In this regard, as a method which allows measuring the predetermined physiologically active substance within a short time, a light scattering method (laser light scattering particle measuring method) in which a mixture liquid of a measurement sample and AL is stirred by using, for example, a magnetic stirring bar to produce gel particles and number of peaks of laser light scattered by the gel particles has been proposed. Similarly, a stirring turbidimetric method in which turbidity of a sample caused by gel particles that are produced by stirring a mixture liquid containing a measurement sample and AL is detected based on intensity of the light transmitted from the mixture liquid has been proposed.

According to the light scattering method, the measurement is performed while the mixture liquid in which a sample and reagents are mixed (i.e., sample liquid) is stirred. In general, by placing a magnetic stirrer under a cuvette and spinning the stirrer, the stirrer bar (stirring bar) which is placed in advance in a sample cuvette is spun to stir the sample. According to Patent Literature 1, the stirring is employed for two purposes. Firstly, it is to generate tiny and even gel particles, and secondly it is to have the gel particles that are produced in a sample pass through the incident laser beam.

However, it is recently reported that the stirring process employed for light scattering method has a risk of causing a significant problem of inducing erroneous measurement for endotoxin measurement. Specifically, according to strong shear stress force occurring between a stirrer and bottom of a cuvette, the proteins contained in a sample or a reagent are self-aggregated (non-specific aggregation) and falsely recognized as a coagulin gel particles that are specific to endotoxin. Even for a case in which the stirrer is floating in the middle of a sample solution and stirred, high shear stress still occurs between the stirrer and the sample solution, and thus the same problem may still exist (see, Non-Patent Literature 1, for example).

Further, according to a mode in which a magnet is spun using a motor and a magnetized stirrer is spun in a sample cuvette, the motor should be placed under the cuvette and plural cuvettes may not be placed close to each other, and therefore a large size apparatus is unavoidable. As such, it is difficult to have many measurable channels in one apparatus.

Endotoxin is a very unstable substance and its activity changes depending on various external causes. Thus, the measurement value easily has a deviation and precise measurement of endotoxin is difficult to achieve. In this connection, studies are made by carrying out the test while confirming the effectiveness of the measurement by performing an addition and recovery test with use of a negative control and a positive control, or the like.

Further, for measurement of a solid sample or a frozen/refrigerated sample, when the time from the dissolution of a sample or warming to room temperature of a sample to the measurement is different, a different measurement value is obtained due to the time dependent change of activity of endotoxin. For avoiding such problems, measurement deviation is generally lowered by measuring simultaneously positive controls with various concentration for obtaining a calibration curve, and a sample to be determined, and a negative control.

According to Japanese Pharmacopoeia, it is required to have three or more types and n=3 or more for a calibration curve reliability test, five types and n=2 or more for a reaction interfering factors test (addition recovery), and four types and n=2 or more for a quantitative test, and therefore an apparatus capable of allowing simultaneous test of many samples is required. In addition, to achieve an apparatus with many channels, it is required to have a small measurement system. In addition, according to a method in which a sample is stirred by spinning a stirrer placed under a cuvette, light is blocked by the stirrer.

Further, according to a conventional apparatus for measuring a predetermined physiologically active substance, laterally)(90° scattered light by the particles gelified in a sample cell is detected (Patent Literature 2). According to this method, only the scattered light, that is, signal light, is detected, and therefore good S/N ratio is obtained. However, as the light amount is insufficient, a measurement system with high sensitivity is required. Further, due to the characteristic construction of an optical system, it takes quite a space, and therefore it is also not suitable for having multichannel. Meanwhile, there is also a method in which gel particle production state is determined by measuring fluctuation in transmitted light based on detection output of transmitted light at the front side of a sample cell and quantifying endotoxin in a sample from the resulting data (Patent Literature 3).

For such case, a light source, a sample cell, and a detector can be arranged along a single axis, and therefore, compared to a case of lateral scattering, it does not take much space even when multichannel is formed. Meanwhile, the technical element which is common in those prior art documents is that a mixture liquid containing a sample and reagents in a sample cell is stirred using a stirring member. In this regard, when a subject substance in a sample (endotoxin) is gelified by reaction with a reagent (AL), the gel stays at the same place in such state, and as a result, the entire reaction occurring in a sample cell may not be recognized with a fixed optical system. Thus, to see the progress level of gelation occurring in an entire sample cell, stirring is an essential technical element. However, it has been recently found that aggregation of proteins caused by stirring has an effect on measurement accuracy. The reason is believed due to the fact that proteins experience the shear stress caused by stirring and aggregation which is not related to the reaction between endotoxin and the AL reagent occurs.

### CITATION LIST

### Patent Literature

PTL1: Japanese Patent No. 4886785
PTL2: PCT International Publication No. WO 2008/038329
PTL3: Japanese Patent No. 4551980
PTL 4: Japanese Patent 2010-216878

### Non-Patent Literature

NPL1: “Problems in Clinical Application of Endotoxin Measurement Using Endotoxin Light Scattering Measurement”, by TAKAHASHI MANABU, et al., Japan journal of critical care for endotoxemia, vol. 14, No. 1, pp. 111-119, 2010.

### SUMMARY OF INVENTION

### Technical Problem

The present invention has been devised in consideration of the aforementioned problems, and an object thereof is, with regard to detection of a physiologically active substance of biological origin and measurement of its concentration in a sample, to provide a technique for highly accurate detection of a physiologically active substance of biological origin and measurement of its concentration with a simple constitution by allowing moving of gel particles that are produced in a sample without using a mechanical stirring member. Further, it is also to provide a technique for reducing a space and allowing multichannel based on measurement of forward scattered light.

### Solution to Problem

As a solution for the problems described above, the most significant characteristic of the invention is firstly to partially heat/cool a sample cell to generate thermal convection in a mixture liquid in a sample cell so as to move gel particles that are produced in the mixture liquid. Further, among the scattered light from a mixture liquid in a sample cell, based on the intensity of forward scattered light component which is scattered in direction of the optical axis of outgoing light opposite side of the sample cell from the optical axis of incident light illuminated from the light source to the sample cell, time series change in the number of gel particles is measured. The characteristic of the invention also includes a cuvette which is made of a heat resistant glass for allowing dry heat sterilization and has sample cells corresponding to plural channels.

More specifically, the characteristic of the invention is to include: a sample cell for retaining a mixture liquid comprising a sample containing a physiologically active substance of biological origin like endotoxin and β-D-glucan and a reagent for inducing gelation with the physiologically active substance of biological origin; light emitting means for illuminating light beam from a light source to the mixture liquid in the sample cell; convection generating means for moving gel particles that are produced in the mixture liquid by partially heating/cooling the sample cell or the mixture liquid in itself and generating thermal convection in the mixture liquid in the sample cell; light detecting means for detecting scattered light which is incident light beam scattered by gel particles that are formed in the sample cell, and measuring means for measuring time series change in the number of gel particles based on the intensity of scattered light detected by the light detecting means.

Accordingly, thermal convection is generated within a mixture liquid, and thus an effect of moving gel particles produced in the mixture liquid so that the gel particles can pass through the incident laser beam more definitely is obtained without having a mechanical stirring member. For such case, it is allowable that heat is unilaterally supplied from a heater to a bottom part of a sample cell. Further, for endotoxin measurement, it is described in Pharmacopoeia that the sample needs to be maintained at 37±1° C. during measurement. Thus, according to the invention, thermal convection is generated in a solution based on, a temperature distribution in a solution to be maintained, or a difference between temperature of a solution to be maintained and temperature of external air to move the gel particles that are produced in the mixture liquid in a sample cell, and as a result, the formed gel particles can more definitely pass through the laser beam. Further, convection generating means may be means for moving the produced gel particles by which temperature of a mixture liquid is maintained at constant temperature by heating/cooling it so that thermal convection is generated in a sample. Further, as used herein, the expression “ . . . by partially heating/cooling a sample cell so that thermal convection is generated within a mixture liquid in the sample cell . . . ” means not only a case in which part of a sample cell is heated/cooled but also a case in which the entire sample cell to be heated/cooled is heated/cooled by a substance having temperature distribution. Further, the expression “time series change in the number of gel particles” described above means a change in the number of gel particles over time.

Further, in the invention, the convection generating means may include means for heating/cooling from the bottom part of the sample cell and/or means for heating/cooling from the top part of the sample cell.

Further, in the invention, the convection generating means may include a heater, and the heater may be in contact with the sample cell and supplies, via a thermistor, heat to a member having a hole at a site through which the light beam from a light source or scattered light passes.

Further, in the invention, the convection generating means may include a heater, and the heater may be an ITO heater with a light transmitting property.

Further, in the invention, temperature measuring means for measuring the temperature of the mixture liquid may be further included. Further, external air temperature measuring means for measuring the temperature of external air may be further included. Further, the time point at which the difference value of the number of gel particles produced in the sample cell per unit time is greater than the threshold value is taken as gelation detection time.

Further, the invention may be a method for measuring a physiologically active substance of biological origin by using the apparatus for measuring a physiologically active substance of biological origin in which the time point at which time the difference value of the number of gel particles produced in the sample cell per unit time is greater than the threshold value is taken as gelation detection time, wherein, by calculating temperature difference between the temperature of the mixture liquid and the temperature of external air based on output of the temperature measuring means and the external air temperature measuring means, velocity of thermal convection occurring in the mixture liquid is calculated and the threshold value is adjusted in accordance with the velocity of thermal convection.

Further, the invention may be a method for measuring a physiologically active substance of biological origin by using the apparatus for measuring a physiologically active substance of biological origin in which the time point at which time the difference value of the number of gel particles produced in the sample cell per unit time is greater than the threshold value is taken as gelation detection time, wherein, by maintaining the temperature difference between the external air temperature and the temperature of a site at which the sample cell is heated/cooled is kept at constant level based on output of the external air temperature measuring means and output of the temperature measurement means, velocity of thermal convection occurring in the liquid is kept constant irrespective of temperature of external air.

Further, the invention may be an apparatus for measuring a physiologically active substance of biological origin by producing a mixture liquid containing an AL reagent, which contains AL as amoebocyte lysate of limulus, and a sample, which contains predetermined physiologically active substance of biological origin, and detecting aggregation or gelation of proteins derived from a reaction between the AL and the physiologically active substance in the mixture liquid to detect the physiologically active substance contained in the sample or measure the concentration of the physiologically active substance, the apparatus comprising; a sample cell for retaining the mixture liquid; light emitting means for illuminating light beam from a light source to the mixture liquid in a sample cell, light detecting means for detecting the light, which is illuminated from the light emitting means, and scattered from the gel particles produced in a liquid mixture, and measuring means for measuring time series change in the number of gel particles based on the intensity of forward scattered light component which is scattered in direction of the optical axis of outgoing light opposite side of the sample cell from the optical axis of incident light illuminated from the light source to the sample cell.

In that case, convection generating means for stirring the mixture liquid by partially heating/cooling the sample cell or the mixture liquid in itself and generating thermal convection in the mixture liquid in a sample cell may be further included.

Further, the measuring means includes a measurement system for detecting output of the forward scattered light component, and the measurement system includes; a first lens system which collects, among the light scattered from the mixture liquid in a sample cell, forward scattered light component which is scattered in direction of the optical axis of outgoing light opposite side of the sample cell from the optical axis of incident light illuminated from the light source to the sample cell, and emits the collected light as parallel light, a transparent plate having a dark spot formed thereon for blocking light components having the same axis as the optical axis of outgoing light included in the parallel light which passes through without being scattered in the mixture liquid, a second lens system for collecting the parallel light not including the light components blocked by the dark spot, a pin hole for allowing partial pass through of the light collected by the second lens system, and a forward scattered light detecting means for detecting the light passed through the pin hole.

Further, the measuring means includes a measurement system for detecting intensity of the forward scattered light component, and wherein the measurement system includes: a third lens system which collects, among the light scattered from the mixture liquid in a sample cell, the forward scattered light component which is scattered in direction of the optical axis of outgoing light opposite side of the sample cell from the optical axis of incident light illuminated from the light source to the sample cell, a pin hole which has been formed outside the axis of the optical axis of outgoing light, for blocking the outgoing light collected by the third lens system and passed through the mixture liquid without being scattered by it, and also for allowing partial pass through of the forward scattered light component collected by the third lens system, and forward scattered light detecting means for detecting the light passed through the pin hole.

In that case, regarding the direction of the optical axis of outgoing light, the pin hole is formed on a site other than light collection point at which the outgoing light passed through the mixture liquid without being scattered is collected by the third lens system.

Further, the measuring means includes a multichannel measurement system, and the light emitting means divides light from a single light source into light for the multichannel and the light is illuminated on the mixture liquid in a plurality of sample cells corresponding to each channel. For the above case, by having a light source plurally split from a single light source by optic fibers or the like for obtaining multichannel, it is unnecessary to perform calibration for compensating deviation among different apparatuses, which occurs when plural light sources are used.

Further, the invention may be a cuvette used for the apparatus for measuring a physiologically active substance of biological origin, wherein the cuvette is formed by comprising heat resistant glass which can be sterilized by dry heat and comprising sample cells corresponding to each channel. In the above case, the sample cells corresponding to each channel may be formed to be in a single row. Further, the sample cells corresponding to each channel may be formed to be in two rows. Further, blocking means for preventing incorporation of scattered light from a mixture liquid in one sample cell to a neighboring sample cell may be installed between the one sample cell and the neighboring sample cell.

According to the invention, the cuvette is made of heat resistant glass which can be sterilized by dry heat, and plural cuvettes are drawn up to respond multichannel measurement. Further, as stirring by spinning of a magnetic stirring rod as employed for a conventional technique is unnecessary, it has a cubic shape instead of a cylinder shape. According to a cylinder shape of a conventional technique, an aberration should be taken into consideration as it has a curved incidence surface for laser. However, for a cubic shape, the incidence surface for laser is flat, and therefore measurement accuracy is improved.

Further, according to the invention, the cuvette may include; an outside cuvette which is formed of a non-light transmitting material and has a plurality of sample cells formed therein and a hole through which light can pass through or a window through which light can transmit is formed at a site which corresponds to center of the sample cell on the lateral surface and/or bottom surface of the cuvette, and inside cuvettes which are formed of heat resistant glass, have external shapes almost the same as the internal shapes of the sample cells, and can be inserted into the sample cells.

According to the constitution described above, it is unnecessary to install blocking means for preventing incorporation of scattered light from a mixture liquid in one sample cell to a neighboring sample cell. Further, use amount of heat resistant glass can be relatively reduced, and thus cost related to an apparatus can be reduced more.

Further, regarding the means for solving the problems of the invention described above, a possible combination thereof can be also employed.

### Advantageous Effects of Invention

According to the invention, for detection of a physiologically active substance of biological origin and measurement of the concentration of the physiologically active substance, gel particles that are produced in a sample can move without using a mechanical stirring member, and thus with a simpler constitution detection and concentration measurement of a physiologically active substance of biological origin can be achieved with high accuracy. Further, as space for measurement can be reduced and multichannel measurement can be achieved by measurement of forward scattered light, automatic measurement expected in future may be promoted. Still further, as the laser incident surface of a cuvette is flat, highly accurate measurement that is hardly affected by aberration can be performed.

### BRIEF DESCRIPTION OF DRAWINGS

FIG. 1 is a diagram illustrating a schematic configuration of a conventional light scattered particle measuring apparatus.

FIG. 2 is a diagram illustrating a schematic configuration of a light scattered particle measuring apparatus relating to Example 1 of the invention.

FIG. 3 is a diagram illustrating a schematic configuration of a light scattered particle measuring apparatus of the second embodiment relating to Example 1 of the invention.

FIG. 4 is a diagram illustrating a schematic configuration of a light scattered particle measuring apparatus of the third embodiment relating to Example 1 of the invention.

FIG. 5 is a diagram illustrating a schematic configuration of a light scattered particle measuring apparatus of the fourth embodiment relating to Example 1 of the invention.

FIG. 6 is a diagram illustrating a schematic configuration of a light scattered particle measuring apparatus of the fifth embodiment relating to Example 1 of the invention.

FIG. 7 is a diagram illustrating a schematic configuration of a light scattered particle measuring apparatus relating to Example 2 of the invention.

FIG. 8 is a diagram illustrating a schematic configuration of a light scattered particle measuring apparatus of another embodiment relating to Example 2 of the invention.

FIG. 9 is a diagram illustrating a schematic configuration of a cuvette relating to Example 3 of the invention. FIG. 9(b) and FIG. 9(c) are Photographic images illustrating schematic configuration of the cuvette.

FIG. 10 is a diagram illustrating a schematic configuration of a cuvette of another embodiment relating to Example 3 of the invention.

FIG. 11 is a diagram illustrating a schematic configuration of a cuvette relating to Example 4 of the invention. FIG. 11(b) and FIG. 11(c) are Photographic images illustrating schematic configuration of the cuvette.

FIG. 12 is a diagram illustrating a schematic configuration of a cuvette relating to Example 5 of the invention.

FIG. 13 is a schematic diagram illustrating a process for gelation of AL by endotoxin or β-D-glucan and a method for detection thereof.

### DESCRIPTION OF EMBODIMENTS

Hereinafter, best modes for carrying out the invention are explained in greater detail in view of the drawings. Further, although in the following examples endotoxin is generally taken as an example of a physiologically active substance of biological origin, it is needless to say that the invention can be applied to other physiologically active substance of biological origin like β-D-glucan.

### Example 1

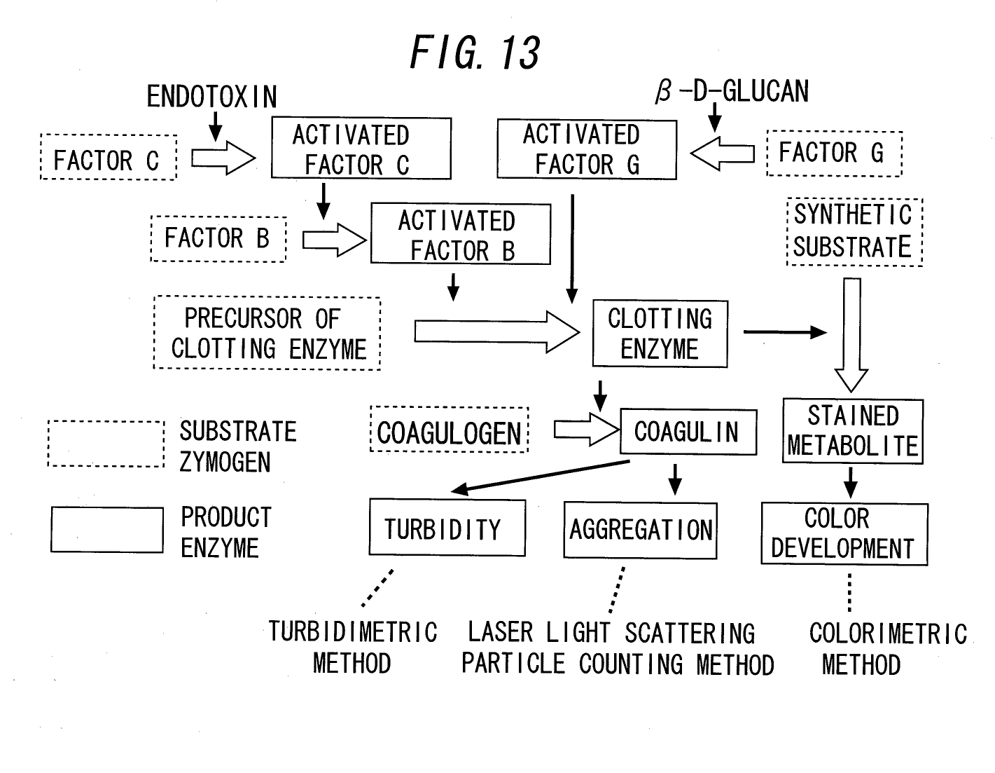

The process of forming a gel by a reaction between AL and endotoxin has been studied well. That is, as illustrated in FIG. 13, when endotoxin is bound to a serine protease, i.e., factor C in AL, the factor C is activated to become activated factor C. The activated factor C hydrolyzes and activates another serine protease, i.e., factor B in AL, and then the factor B is activated to become activated factor B. The activated factor B immediately hydrolyzes a precursor of clotting enzyme in AL to form clotting enzyme, and further the clotting enzyme hydrolyzes a coagulogen in AL to generate coagulin. Thus, it is believed that the generated coagulins are then associated with each other to further form an insoluble gel, and the whole AL is involved in the formation to turn into a gel.

In addition, when β-D-glucan is bound to factor G in AL, the factor G is similarly activated to become activated factor G. The activated factor G hydrolyzes a precursor of clotting enzyme in AL to produce clotting enzyme. As a result, as is the case with the reaction between endotoxin and AL, coagulin is generated, and the generated coagulins are associated with each other to further generate an insoluble gel.

The series of reactions as described above is similar to the process of forming a fibrin gel via serine proteases such as Christmas factor or thrombin present in mammals. Such enzyme cascade reactions have a very strong amplification effect because even a very small amount of an activation factor activates the subsequent cascade in a chain reaction. Therefore, by using the method of measuring a predetermined physiologically active substance using AL, it is possible to detect a very small amount like sub picogram/mL order of the predetermined physiologically active substance.

Measuring methods, which are capable of quantifying predetermined physiologically active substance, include a turbidimetric method and a laser light scattered particle measuring method as described above. As illustrated in FIG. 13, any of these measuring methods detects an aggregated product of coagulins generated by the enzyme cascade reaction of AL, as the turbidity of a sample in the case of the former and as gel fine particles generated in the system in the case of the latter. Thus, a highly sensitive measurement can be achieved.

In particular, as being able to measure directly the microparticles of gel produced in a system, the laser light scattered particle measuring method has higher sensitivity than the turbidimetric method, and as a sample generally consisting of AL and a specimen is forcefully stirred, gel production can be detected within shorter time compared to the turbidimetric method.

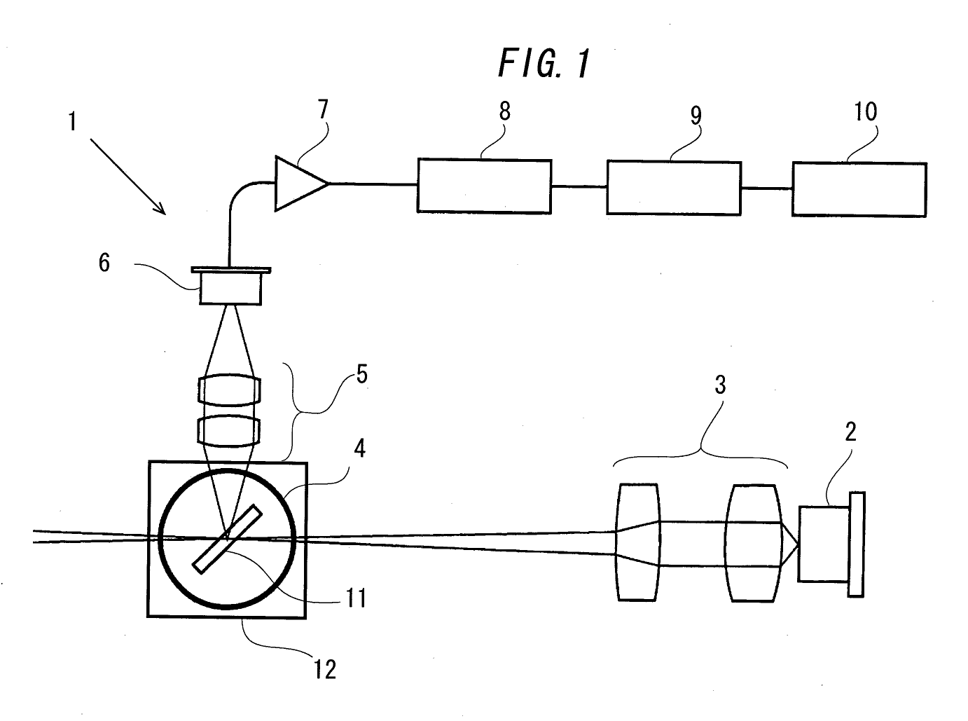

In FIG. 1, a schematic configuration of a conventional light scattered particle measuring apparatus 1 as an apparatus for endotoxin measurement is illustrated. A light source 2 used in the light scattered particle measuring apparatus 1 is a laser light source. Alternatively, it may be a super-high-luminance LED or the like. Light irradiated from the light source 2 is concentrated by an incidence optical system 3 and then incident on a sample cell 4. The sample cell 4 retains a mixture liquid containing a sample for endotoxin measurement and an AL reagent. Light incident on the sample cell 4 is scattered by particles (measuring objects, such as coagulin monomers and coagulin oligomers) in the liquid mixture.

An emitting optical system 5 is arranged on the lateral side of an incident optical axis of the sample cell 4. In addition, a light detecting element 6 is arranged on the extended line of the optical axis of the emitting optical system 5. Here, the light detecting element 6 is provided for detecting scattered light, which is scattered by particles in the mixture liquid in the sample cell 4 and concentrated by the emitting optical system 5, and converting the detected light into an electric signal. The light detecting element 6 is electrically connected to an amplifying circuit 7 for amplifying the electric signal photoelectrically converted by the light detecting element 6; a filter 8 for removing a noise from the electric signal amplified by the amplifying circuit 7; an arithmetic unit 9 for calculating the number of gel particles from the number of peaks of the electric signal after the noise removal, determining gelation detection time, and deriving the concentration of endotoxin; and a display unit 10 for displaying results.

Furthermore, the sample cell 4 is provided with a stirring bar 11 for stirring a mixture liquid as a sample, where the stirring bar 11 can be rotated by receiving an electromagnetic force from the outside. A stirrer 12 is arranged on the outside of the sample cell 4. Thus, the presence or absence of stirring and the rate of stirring can be regulated by them.

According to light scattered particle measuring apparatus 1, the appearance time of coagulin gel particles (i.e., gelation detection time=gelation time) as a final step of limulus reaction is measured, and by using calibration relationship established between endotoxin concentration and gelation detection time, concentration of endotoxin in a specimen is calculated.

According to the light scattered particle measuring method using the conventional light scattered particle measuring apparatus 1, the measurement is performed while the mixture liquid in which a sample and reagents are mixed is stirred. In general, as described above, by placing a magnetic stirrer 12 under a sample cell 4 and spinning the stirrer, the stirring bar 11 which is placed in advance in a sample cell 4 is spun to stir the sample. The stirring is employed for two purposes. Firstly, it is to generate tiny and even gel particles, and secondly it is to have gel particles that are formed in a sample pass through the incident laser beam.

However, it is recently reported that the stirring process employed for the light scattered particle measuring method has a risk of causing a significant problem with inducing erroneous measurement for endotoxin measurement. Specifically, according to high shear stress occurring between a stirring bar 11 and bottom of the sample cell 4, the proteins contained in a sample or a reagent are self-aggregated (i.e., non-specific aggregation) and falsely recognized as a coagulin gel particles that are specific to endotoxin. Even for a case in which the stirring bar 11 is floating in the middle of a sample solution and stirred, high shear stress still occurs between the stirring bar 11 and the sample solution, and thus the same problem may still exist.

Further, according to a mode in which a magnet is spun using a motor and a magnetized stirring bar 11 is spun in a sample cell 4, the motor should be placed under the sample cell 4 and plural sample cell 4 cannot be placed close to each other, and therefore a large size apparatus is unavoidable. Thus, it is inconvenient in that having measurable channel number as many as possible in one apparatus is difficult to achieve.

Endotoxin is a very unstable substance and its activity changes depending on various external causes. Thus, the measurement value easily has a deviation and precise measurement of endotoxin is difficult to achieve. In this connection, studies are made by carrying out the test while confirming the effectiveness of the measurement by performing an addition and recovery test with use of a negative control and a positive control, or the like.

Further, for measurement of a solid sample or a frozen/refrigerated sample, when the time from the dissolution of a sample or warming to room temperature of a sample to the measurement is different, a different measurement value is obtained due to the time dependent change of activity of endotoxin. For avoiding such problems, measurement deviation is generally lowered by measuring simultaneously positive controls with various concentration for obtaining a calibration curve, and a sample to be determined, and the negative control.

According to Japanese Pharmacopoeia, it is required to have three or more types and n=3 or more for a calibration curve reliability test, five or more types and n=2 or more for a reaction interfering factors test (addition recovery), and four or more types and n=2 or more for a quantitative test, and therefore an apparatus capable of allowing simultaneous test of many samples is required. In other words, an apparatus having many channels is needed more than ever before.

To achieve an apparatus with many channels, it is required to have a small measurement system. In addition, for a conventional method in which a sample is stirred by spinning the stirring bar 11 under the bottom of the sample cell 4 like the light scattered particle measuring apparatus 1, a motor should be placed under the sample cell 4, and therefore it is difficult to achieve miniaturization. Further, as light is blocked by the stirring bar 11, the light incident direction is limited and freedom for responding to miniaturization and multichannel is lowered.

Thus, according to the present example, the mixture liquid containing a sample and an AL reagent is not mechanically stirred like a stirrer 12. Instead, thermal convection is generated by having temperature gradient in a mixture liquid in the sample cell 4 and gel particles that are produced in the mixture liquid are moved by the thermal convection.

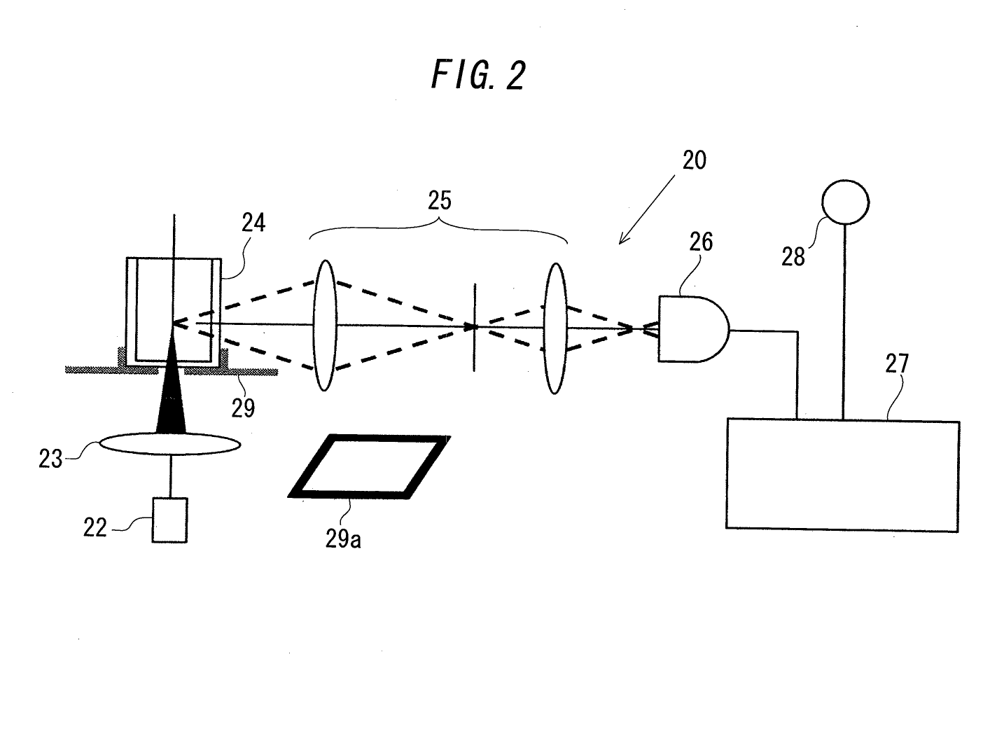

FIG. 2 is a diagram schematically illustrating a configuration of a light scattered particle measuring apparatus 20 according to the present embodiment. A light source 22 used in the light scattered particle measuring apparatus 20 of FIG. 2 is a laser light source. Alternatively, it may be a super-high-luminance LED or the like. Light irradiated from the light source 22 is concentrated by an incidence optical system 23 and then incident on a sample cell 24, in which the incidence optical system 23 corresponds to the light emitting means and is placed under the bottom surface (i.e., lower side) of the sample cell 24. Light incident on the sample cell 24 is scattered by particles (measuring objects, such as coagulin monomers and coagulin oligomers) in the liquid mixture.

An emitting optical system 25 is arranged on the lateral side of an incident optical axis of the sample cell 24. In addition, a light detecting element 26 as the light detecting means is arranged on the extended line of the optical axis of the emitting optical system 25. Here, the light detecting element 26 is provided for detecting scattered light, which is scattered by particles in the mixture liquid in the sample cell 24 and concentrated by the emitting optical system 25, and converting the detected light into an electric signal. The light detecting element 26 is electrically connected to a signal processing unit 27 as the measuring means. The signal processing unit 27 performs amplifying, A/D converting and noise removing of the electric signal photoelectrically converted by the light detecting element 6. The signal processing unit 27 also calculates the number of gel particles from the number of peaks of the electric signal after the noise removal, derives the concentration of endotoxin by determining a gelation detection time, and displays the result. With respect to the signal processing unit 27, more specifically, the time point at which the difference value of the number of gel particles produced in the sample cell 24 per unit time is greater than the threshold value is taken as gelation detection time, and by using the calibration curve data representing the relation between the gelation detection time and endotoxin concentration, the endotoxin concentration is obtained. In the examples given below, the endotoxin concentration can be also obtained by using the same method.

Further, on the sample cell 24, a heater 29 which is placed to touch the bottom surface of the sample 24 and has a hole at a site through which incident light passes is placed (in this regard, it is also possible that, instead of having a hole at a site through which incident light passes, the heater 29 may have an ITO heater 29a having a light transmitting property). Further, when electric current is applied to the heater 29, only the region near the bottom of the sample cell 24 is heated, and thus the mixture liquid near the bottom is heated and moves to the upper side. Then, since the mixture liquid moved to the upper side is cooled by external air, it moves down to the bottom side after cooling. According the repetition of those processes, convection occurs in the mixture liquid, and as a result, the gel particles that are produced in the mixture liquid are moved. According to the present example, the heater 29 corresponds to the convection generating means.

The light scattered particle measuring apparatus 20 is equipped with an external air temperature sensor 28 as the external air temperature measuring means. Further, the sample cell 24 is equipped with a temperature sensor as the temperature measuring means for measuring the temperature of a mixture liquid (not illustrated). Herein, when the gelation detection time is obtained from the difference value of the number of gel particles produced in the sample cell 24 per unit time, the time point at which the difference value is greater than the threshold value is found as gelation detection time. In this example, with respect to the threshold value, it is possible that the temperature difference between the temperature of a mixture liquid and the temperature of external air is calculated from the output of the temperature sensor and the external air temperature sensor 28, velocity of thermal convection occurring in the mixture liquid is calculated from the temperature difference, and the threshold value is adjusted depending on the resulting velocity of thermal convection. In other words, although it is possible to assume that the moving rate of the gel particles is different because the velocity of thermal convection varies depending on the difference between the temperature of external air and the temperature of mixture liquid, by adjusting the threshold value depending on the velocity of thermal convection, influence of the velocity of thermal convection on gelation rate can be cancelled, and therefore measurement with higher accuracy can be achieved. Meanwhile, when an algorithm for recognizing the gelation detection time by using software is used, the external air temperature sensor 28 is unnecessary.

Herein, it is known that the convection velocity at the velocity boundary layer which is present near a boundary between convecting fluid and external air can be expressed with the following formula (1)

$$\begin{matrix}[ Math . 1 ] & \\ \frac{u ( y )}{{u}_{∞}} = \frac{3}{2} ( \frac{y}{δ} ) - \frac{1}{2} {( \frac{y}{δ} )}^{3} & ( 1 )\end{matrix}$$

In the formula (1), u(y) represents convecting velocity at a site which is distance y-away from the boundary between the fluid and external air. Further, 6 represents thickness of the velocity boundary layer. When temperature of fluid within the boundary layer is T1 (=temperature of mixture liquid) and temperature of cuvette wall is T2 (=temperature of external air), δ is a function of (T1−T2). Further, u_{∞} represents the convection velocity at a site which is at least δ away from the boundary, in which δ is the thickness of velocity boundary layer.

Specifically, according to the light scattered particle measuring apparatus 20, the external air temperature T2 is detected by the external air temperature sensor 28 and T1 is predicted from the temperature of the heater 29. Further, from T1 and T2, the convection velocity u (x1, y1) at an observation point is predicted. Herein, x1 represents the coordinate which is in parallel direction with the boundary of an observation point and y1 represent the coordinate which is in perpendicular direction with the boundary of an observation point. Further, since it is believed that the number of particles pass through the laser beam per unit time is proportional to u (x1, y1) at an observation point, the aforementioned threshold value may be also corrected to be proportional to the value n.

Further, in the present example, it is also possible that the temperature difference between the temperature of a mixture liquid and the temperature of external air is calculated from the output of the temperature sensor and the external air temperature sensor 28, velocity of thermal convection occurring in the mixture liquid is calculated from the temperature difference, and the temperature (heat generation amount) of the heater 29 (or ITO heater 29a) is controlled to have constant velocity of thermal convection. Accordingly, an influence of the velocity of thermal convection on gelation rate can be inhibited, and therefore measurement with higher accuracy can be achieved. Further, for a case in which the temperature difference between the temperature of a mixture liquid and temperature of external air is calculated and the velocity of thermal convection occurring in the mixture liquid is calculated from the temperature difference, the formula (1) can be similarly used as described above.

According to the present example, the mixture liquid is not stirred mechanically with the stirring bar 11, and thus false recognition of the self-aggregation (non-specific aggregation) of the proteins contained in a sample or a reagent, which is caused by strong shear stress force occurring between the stirring bar 11 and bottom of the sample cell 4, as endotoxin-specific coagulin gel particles can be inhibited.

Further, according to the present example, the bottom surface of the sample cell is not covered by a stirrer or a stirring bar, thus incident light can be applied to the bottom side of a sample cell. Accordingly, by arranging plural systems illustrated in FIG. 2, multichannel measurement can be performed. In the example of FIG. 2, plural light sources 22 may be provided to have one light source for each channel.

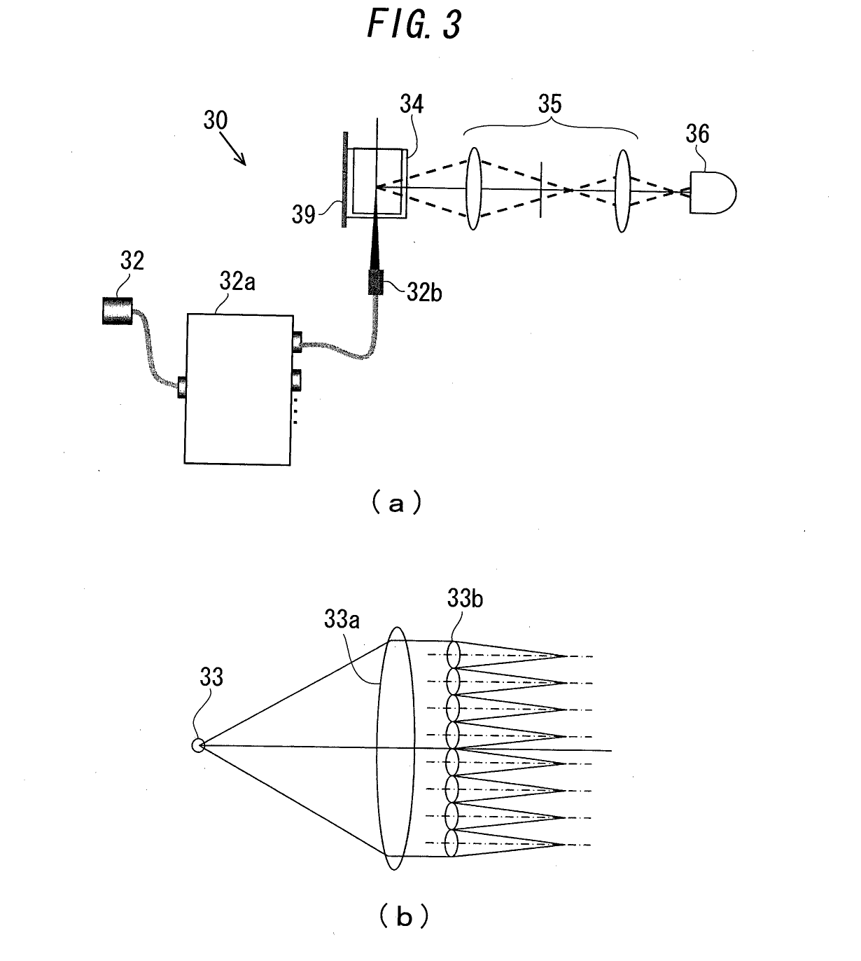

In FIG. 3(a), a light scattered particle measuring apparatus 30 is illustrated as another embodiment of the example. According to the example, a heater 39 is installed to be in contact with side surface of a sample cell 34. It is unnecessary for the heater 39 to have a hole for incident light and instead of using a special heater like an ITO heater, a common heater like a thermistor may be used. Further, in the light scattered particle measuring apparatus 30, a light source 32 is a fiber pigtailed LD. The outgoing light from the light source 32 is supplied to a 1:8 fiber coupler 32a, and diverged to eight. The fiber diverged to eight is connected to eight SELFOC condenser lenses 32b.

Further, the light scattered particle measuring apparatus 30 is equipped with eight measurement systems, each of which includes the sample cell 34, the emitting optical system 35, and the light detecting element 36. Accordingly, eight-channel measurement can be achieved. Further, in the light scattered particle measuring apparatus 30, it is also possible that the light source is a Laser Diode 33, and outgoing light from a light source 33 is converted to parallel light by a collimator lens 33a and divided into eight by a micro lens array 33b as illustrated in FIG. 3(b). When the light scattered particle measuring apparatus 30 is equipped with eight measurement systems each of which includes the sample cell 34, the emitting optical system 35, and the light detecting element 36, eight-channel measurement can be also achieved.

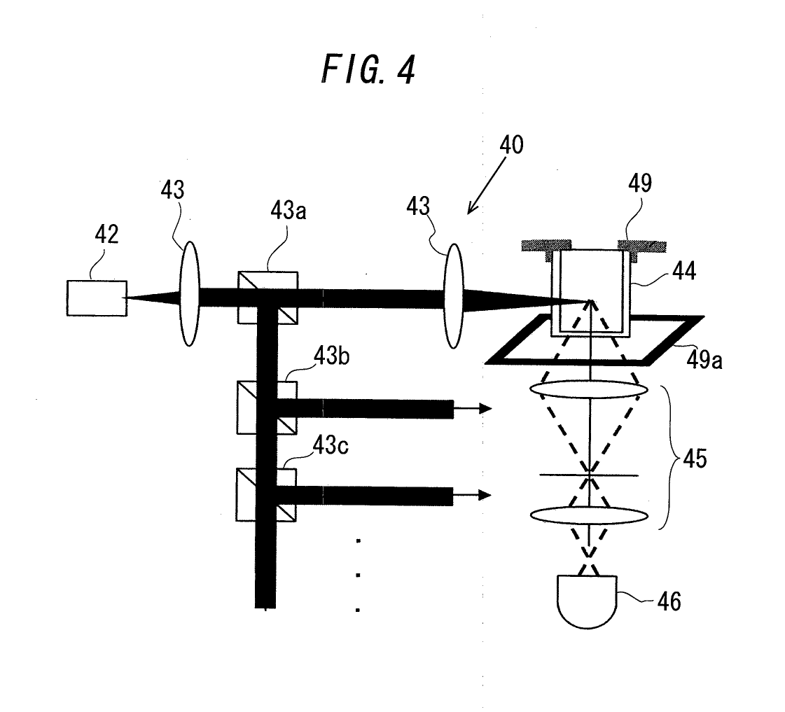

Next, brief construction of a light scattered particle measuring apparatus 40 as the third embodiment of the example is illustrated in FIG. 4. Characteristics of the light scattered particle measuring apparatus 40 reside in that illuminating light from a light source 42 is plurally diverged with use of half mirror 43a, 43b, 43c, . . . and the like. In addition, the diverged incident light is incident on a sample cell 44 which is installed in response to each incident light. The light scattered particle measuring apparatus 40 is equipped with measurement systems, each of which includes the sample cell 44, an emitting optical system 45, and a light detecting element 46, the number of which is same as the divergence number of incident light. Accordingly, multichannel measurement can be performed.

Further, the light scattered particle measuring apparatus 40 is equipped with a thermistor 49 and an ITO heater 49a, above and below the sample cell 44, respectively. By heating the mixture liquid from both top and bottom sides, temperature of the mixture liquid can be maintained at average temperature of 37 degrees, and by efficiently generating thermal convection, the gel particles in the mixture liquid can be moved efficiently. Further, in this example, by controlling the temperature difference between the thermistor 49 and the ITO heater 49a, it becomes possible to control the velocity of thermal convection. In this regard, since thermal convection is generated by temperature difference between two heat sources irrespective of external air temperature, the moving rate of the gel particles in the mixture liquid can be controlled with higher accuracy without being affected by the external air temperature.

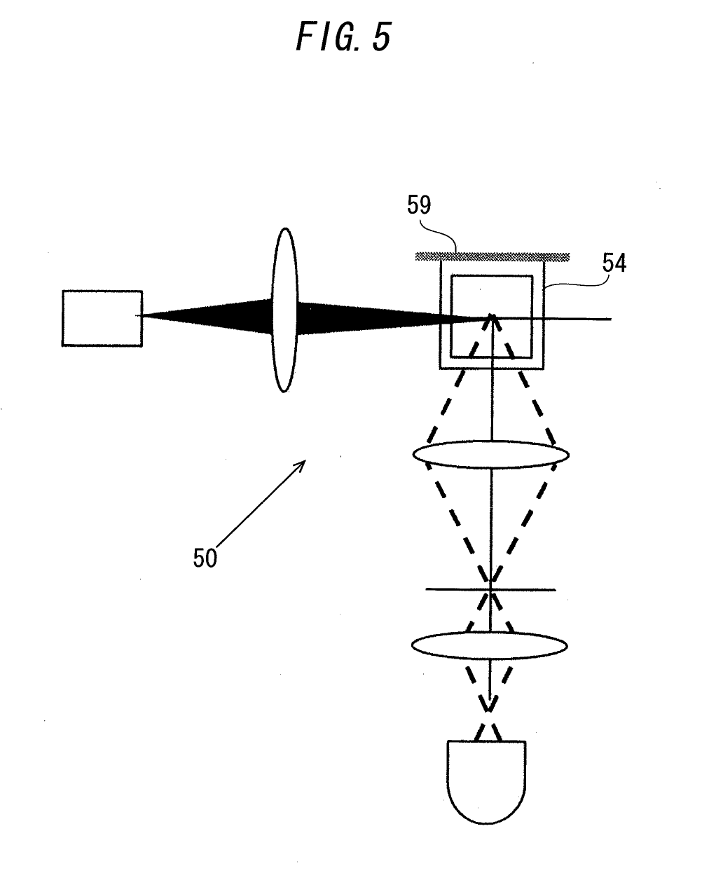

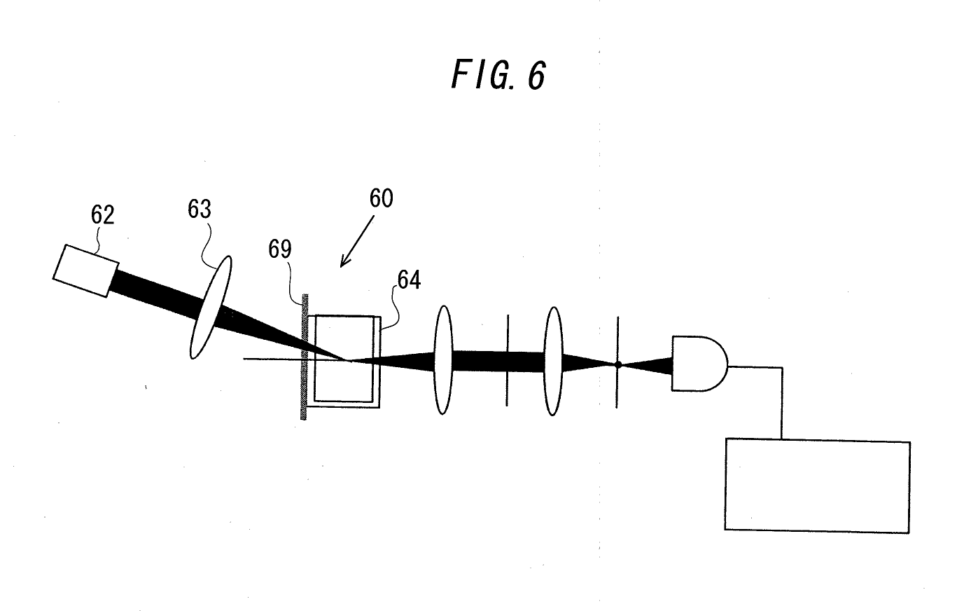

In FIG. 5, a light scattered particle measuring apparatus 50 is illustrated as the fourth embodiment of the example. According to the embodiment, a heater 59 like a thermistor having no hole for light transmission is in contact with a sample cell 54. In FIG. 6, a light scattered particle measuring apparatus 60 is illustrated as the fifth embodiment of the example. According to the embodiment, a heater 69 having a hole for light transmission is in contact with a sample cell 64. Further, a light source 62 and an incidence optical system 63 are arranged in tilted direction compared to the optical axis of outgoing light.

Further, although a case in which thermal convection is generated within a mixture liquid in a sample cell by heating the sample cell with a heater is explained in the above example, it is also possible to have thermal convection generated within a mixture liquid in a sample cell by partial cooling of a sample cell using a refrigerating unit like Peltier element or a water cooling device. Further, in above mentioned Example, the light scattered particle measuring apparatus is equipped with heaters such as thermistors or ITO heaters outside of the sample cell, but the light scattered particle measuring apparatus can be equipped with heaters inside of the sample cell. In that case, heaters partially heat the mixture liquid in itself and generating thermal convection in the mixture liquid in the sample cell.

### Example 2

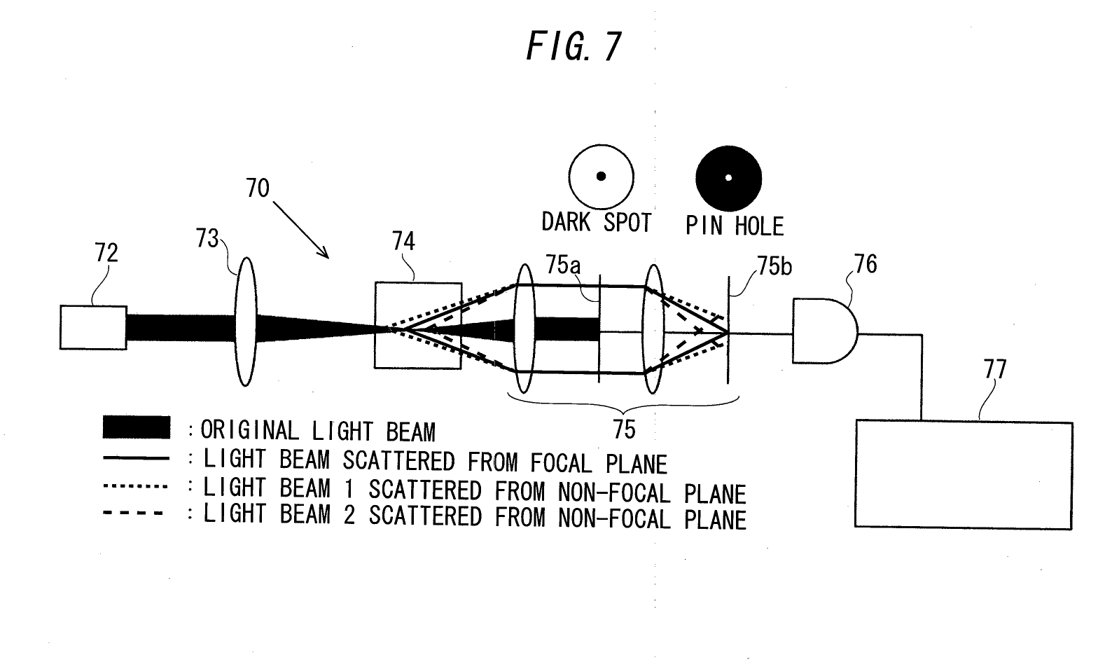

Next, brief construction of a light scattered particle measuring apparatus 70 relating to Example 2 of the invention is illustrated in FIG. 7. In FIG. 7, light illuminated from a light source 72 used for the light scattered particle measuring apparatus 70 is concentrated by an incidence optical system 73 and incident on a sample cell 74. The incident light on the sample cell 74 is scattered by particles in a mixture liquid (subject for measurement like coagulin monomer and coagulin oligomer).

With regard to the sample cell 74, an emitting optical system 75 is placed in front of optical axis of incident light (i.e., on the extended line of optical axis of incident light). Scattered light is converted to parallel light by a first lens system, which is a front lens system of the emitting optical system 75. The parallel light is condensed by a second lens system, which is the last lens system of the emitting optical system 75. On the extended line of the optical axis of the emitting optical system 75, a light detecting element 76 is placed as the forward scattered light detecting means, which detects the light scattered by particles in a mixture liquid in the sample cell 74 and concentrated by the emitting optical system 75 and converts the light to an electric signal. To the light detecting element 76, a signal processing unit 77, which is to amplify the electric signal photoelectrically converted by the light detecting element 76, carry out A/D conversion, and remove a noise, is connected. Further, the signal processing unit 77 calculates the number of gel particles from the number of peaks of the electric signal after noise removal, yield concentration of endotoxin by determining gelation detection time, and display the results.

Herein, the incident light which passes through the sample cell 74 without being scattered by particles in a mixture liquid is blocked by a dark spot plate 75a as a transparent plate having a dark spot formed thereon in the emitting optical system 75. In the dark spot plate 75a, a dark spot is formed by black coloration only at an area of a transparent plate through which the incident light after passing through the sample cell 74 passes. Thus, the incident light passed through the sample cell 74 is blocked by a dark spot. Meanwhile, the light scattered from the sample cell 74 passes through a transparent area around the dark spot.

Further, at final stage of the emitting optical system 75 (i.e., in front of a second lens system), a pin hole plate 75b is formed on a condensation point for light which passes through an observation point. Only the light scattered on the focal plane (i.e., observation point) of incident light beam of the sample cell 74 is collected on the hole of the pin hole plate 75b. As a result, the light scattered on the focal plane (i.e., observation point) of incident light beam of the sample cell 74 mainly passes through the hole of pin hole plate 75b and is detected by the light detecting element 76. Thus, according to the present example, it is possible to primarily select and detect scattered light from a focal plane (i.e., observation point) of incident light beam and the incident light passed through the sample cell 74 can be removed, and therefore measurement accuracy can be enhanced. In addition, by having the emitting optical system 75 of FIG. 7, the measurement system of the example is constructed.

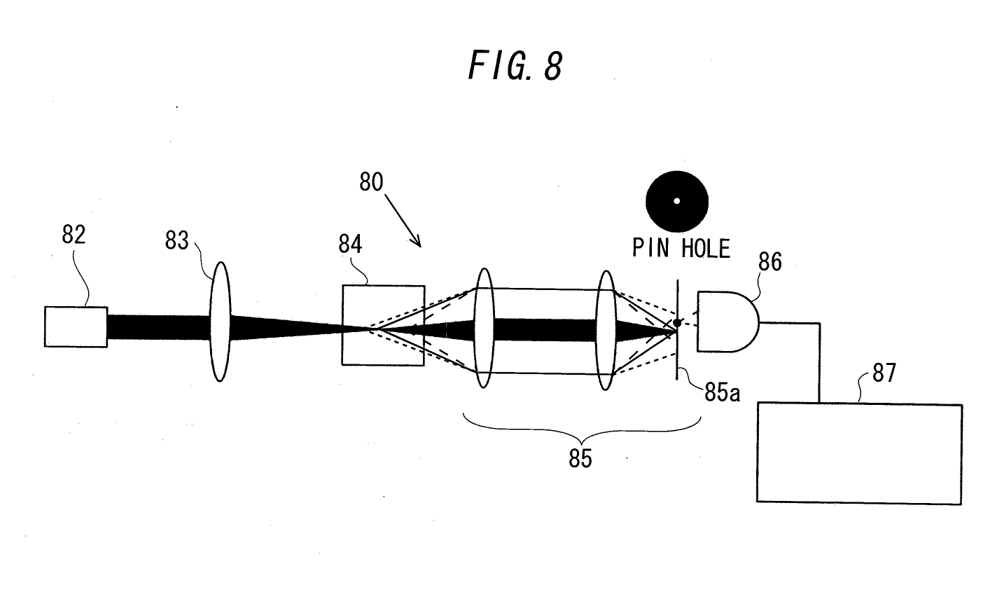

Brief construction of a light scattered particle measuring apparatus 80 as another embodiment of the present example is illustrated in FIG. 8. According to the embodiment, a hole of a pin hole plate 85a is open in an area outside the emitting optical axis. Thus, the incident light passed through a sample cell 84 is blocked in an area other than the hole of the pin hole plate 85a, and the light scattered before and after the focal plane (observation point) of incident light beam passes through the hole of the pin hole plate 85a. As a result, the incident light passed through the sample cell 84 can be removed with fewer components, and thus the measurement accuracy can be enhanced. Also, according to the present embodiment, the measurement system is constituted by having an emitting optical system 85, and a first lens system as a front lens system and a second lens system as a last lens system in the emitting optical system 85 have the functions equivalent to the first lens system and second lens system of the emitting optical system 75. According to the emitting optical system 85 of the present embodiment, it is unnecessary to convert outgoing light to parallel light first. Thus, the first lens system and second lens system can be achieved by a single lens system. By having such constitution, the number of components can be further reduced.

As seen from FIG. 7 and FIG. 8, among the scattered light from the mixture liquid of a sample cell, time series change in the number of gel particles is measured based on the intensity of forward scattered light component which is scattered in direction of the optical axis of outgoing light opposite side of the sample cell from the optical axis of incident light illuminated from the light source to the sample cell (i.e., direction of the optical axis of outgoing light which is on the extended line of the optical axis of incident light after passing through the sample cell). Accordingly, since it is possible to arrange plural sample cells and measurement systems vertical to the optical axis, multichannel measurement can be simultaneously performed without needing a large size apparatus. Thus, it becomes possible to measure many samples with higher efficiency.

Further, according to a measurement system illustrated in FIG. 8 in which the incident light passed through the sample cell is blocked by the pin hole plate 85a without using a dark spot plate and the scattered light is selectively detected, the pin hole plate 85a along the optical axis may be placed on an area which is within a defocusing plane (i.e., a plane which is vertical to the optical axis and on an area which is away from the plane involving the light condensing point in the direction of the optical axis), instead of having it on a plane involving the condensing point of the incident light beam passed through the focal point (i.e., observation point) of incident light beam. Additionally, instead of having the hole of the pin hole plate 85a on the position explained above, a photodetector like photodiode may be directly placed.

According to the arrangement method described above, scattered light of laser beam which entered to the sample cell can be collected from a broader region (i.e., all regions) before and after the observation point, and thus, even when the gel particles randomly produced by gelation pass through any area at any side of laser beam, detection can be successfully made. Herein, when it is desired to capture scattered light from almost every region of laser light beam in the sample cell by using a side scattering arrangement as employed for a conventional laser light scattered particle measuring method, the photodetector needs to cover the entire laser light beam passing through the sample cell, and as a result, the S/N ratio may be lowered. This leads to less merit of a side scattering optical system which claims to maintain high S/N ratio.

### Example 3

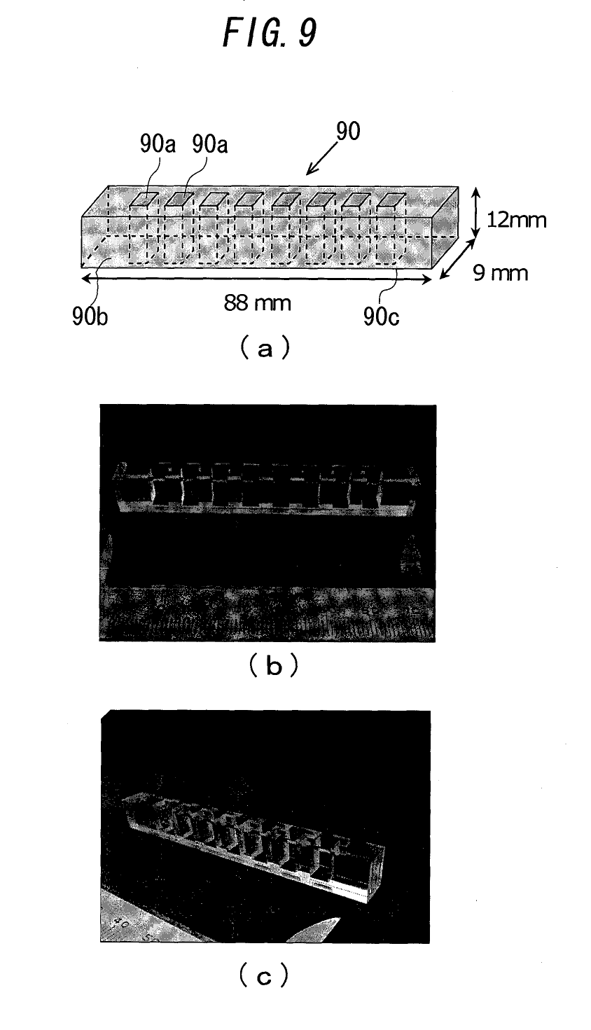

Next, Example 3 of the invention is explained in view of FIG. 9. The present example relates to an exemplary structure of a sample cell (i.e., cuvette) having eight holes (i.e., wells) formed side by side in glass. In the present example, a cuvette 90 made of rectangular parallelepiped glass has eight wells 90a, which also have a rectangular parallelepiped shape as illustrated in FIG. 9(a). According to the structure, in the light scattered particle measuring apparatus having eight channels, light can be incident on a mixture liquid of a sample and AL reagent retained in each well 90a while maintaining as much as possible the state of incident light beam. According to the present example, the number of wells in the cuvette 90 may be eight or more. It may be freely determined depending on use, for example, it may be 16 or 24. As for the glass, heat resistant glass may be used to allow sterilization by dry heat.

Only a side surface 90b and a bottom surface 90c of the cuvette 90 (i.e., only an area through which light passes) are treated to have a mirror surface. The shape of the hole may be freely determined, but preferably a rectangular parallelepiped shape. Photographic image of the cuvette of the present example is illustrated in FIG. 9(b) and FIG. 9(c). To avoid any interference or influence on signal caused by insertion of scattered light from one well 90a to an optical pathway of neighboring channel, a “divider” which is made of a material capable of absorbing/blocking light may be provided between the well 90a and another well 90a. A notch for adding a divider may be preferably formed between the well 90a and another well 90a of the cuvette 90.

Further, according to the present example, distance between the centers of neighboring wells 90a is the same as the distance between holes of a conventional microplate (i.e., 9 mm: gap between holes is determined by ANSI/SBS 2004-1, for Microplates—Footprint Dimensions, for example). By doing so, a probe of a robot for a conventional microplate can be used as it is, and it may be easily applied to an automatic dispenser using a robot. Thus, by using the cuvette of the present example, automatic measurement of endotoxin concentration is possibly promoted.

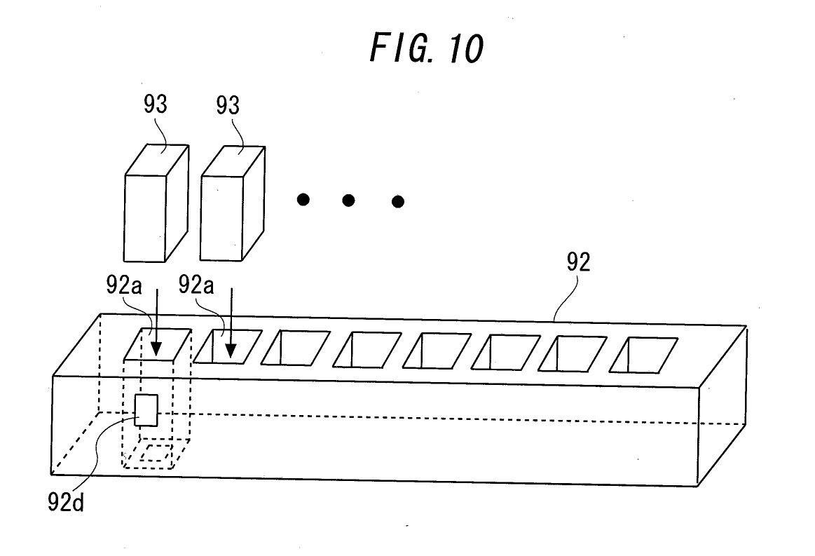

According to the present example, it is also possible to have a constitution illustrated in FIG. 10, in which plural well 92a having a rectangular parallelepiped shape are formed in a row in a cuvette 92 having a rectangular parallelepiped shape which is made of a non-light transmitting material and absorbs and blocks light, and individual glass cuvette 93 which precisely fits in the well 92a is inserted thereto. In this case, it is necessary to have a horizontal hole or vertical hole 92d for transmitting light on a side surface of the cuvette 92, on the place which corresponds to the center of each well 92a. However, the divider described above would be unnecessary. Further, as the use amount of heat resistant glass can be relatively lowered, cost related to the apparatus can be further reduced.

According to the present example, the cuvette 92 corresponds to an outside cuvette and the cuvette 93 corresponds to an inside cuvette. Further, the well 92a corresponds to a sample cell. According to the present example, the well 92a is formed to be in a single row. However, it may be formed to be in two rows or formed to be in other array mode.

### Example 4

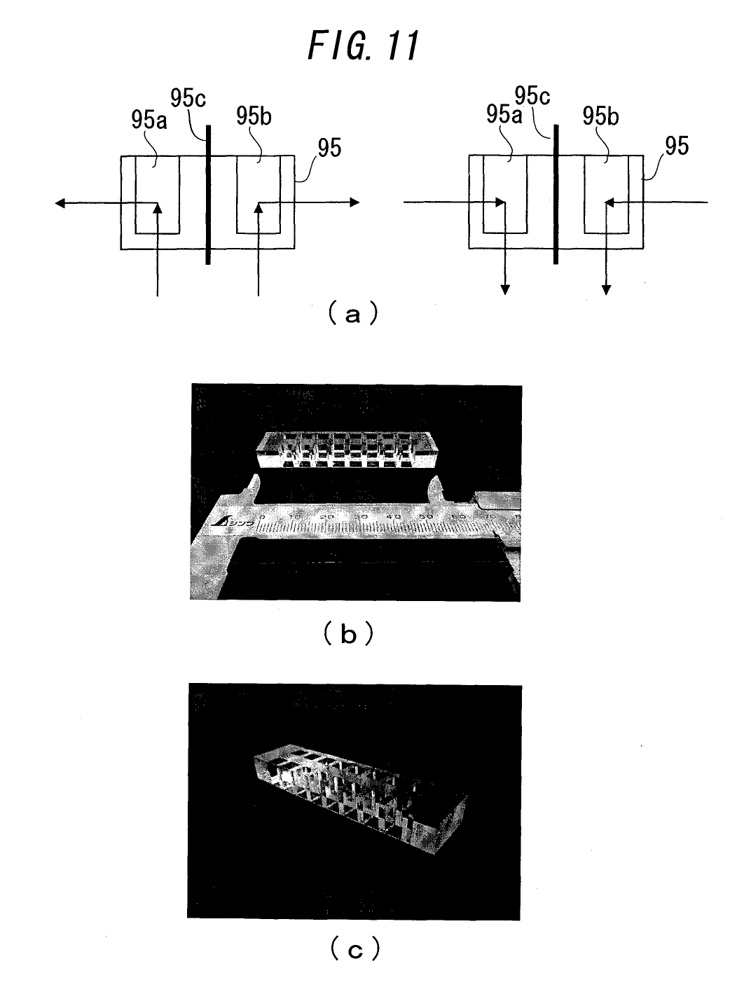

Next, Example 4 of the invention is explained in view of FIG. 11. The present example is an example for eight consecutive cuvettes in two rows in which wells are formed in two rows. FIG. 11(a) is a cross sectional view from the longitudinal direction of a cuvette 95. In the cuvette 95, wells 95a and wells 95b are formed in two rows. For a case of having two rows, in order to prevent interference between neighboring rows, it is necessary to insert a divider 95c for absorbing/blocking light between rows of well to separate them. Further, for a case of two rows like the present example, as illustrated in the left and right diagrams of FIG. 11(a), a method for selecting an optical pathway for incident light and outgoing light may have line symmetric relation between two rows. The left diagram of FIG. 11(a) is an example in which light is illuminated from the bottom of the cuvette 95 and scattered light emitting from the side surface is detected. The right diagram of FIG. 11(a) is an example in which light is illuminated from the side of the cuvette 95 and scattered light emitting from the bottom surface is detected. In FIG. 11(b) and FIG. 11(c), photographic images of the eight consecutive cuvettes in two rows of the present example are given.

### Example 5

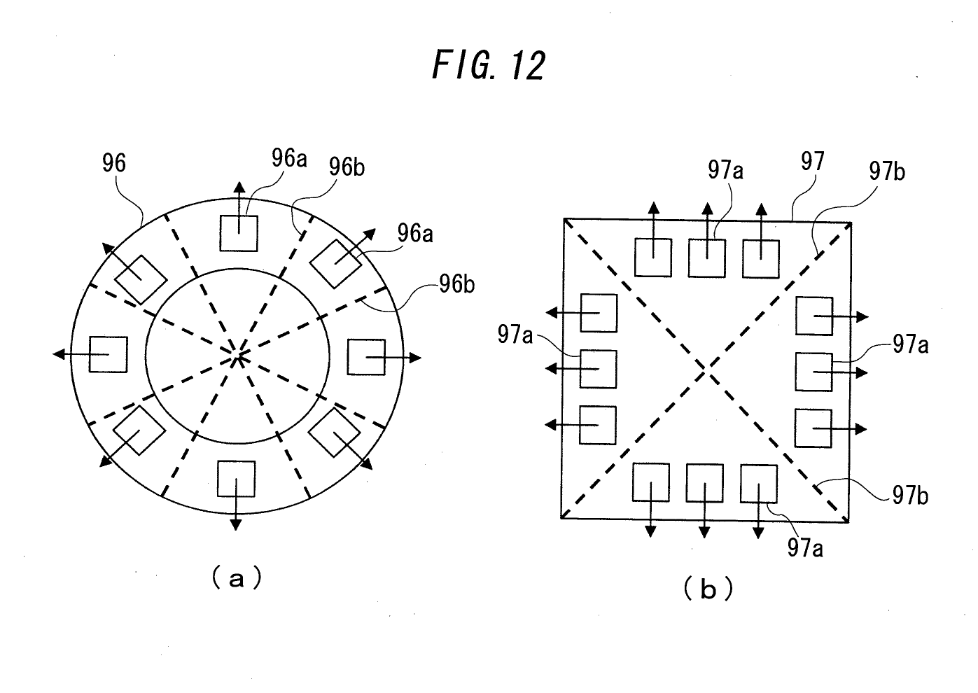

Next, Example 5 of the invention is explained in view of FIG. 12. The present example is an example in which plural wells are formed around periphery of a cuvette having a cylinder shape or a rectangular parallelepiped shape. FIG. 12(a) illustrates a plain view of a cuvette 96 with a cylinder shape. Near the periphery of the cuvette 96, rectangular wells 96a are formed side by side in a concentric circle shape so that one of its side surfaces faces the outside in diameter direction. In addition, in order to prevent interference between neighboring wells 96a, a divider 96b is inserted radially between the wells 96a for absorbing/blocking light. Further, according to FIG. 12(a), incident light falls from the bottom of the cuvette (i.e., longitudinally inward of paper surface) or the top of the cuvette (i.e., longitudinally front of paper surface) and the outgoing light scattered radially to the periphery is detected.

Further, FIG. 12(b) illustrates a plain view of a cuvette 97 with a rectangular parallelepiped shape. Near the periphery of the cuvette 97, rectangular wells 97a are formed side by side such that one of its side surfaces is parallel to each side surface of the cuvette 97. In addition, in order to prevent interference between neighboring wells, a divider 97b is inserted diagonally in the cuvette 97, for example. Further, also in FIG. 12(b), incident light falls from the bottom of the cuvette (i.e., longitudinally inward of paper surface) or the top of the cuvette (i.e., longitudinally front of paper surface) and the outgoing light scattered perpendicularly to the side of the cuvette is detected.

### REFERENCE SIGNS LIST

1, 20, 30, 40, 50, 60, 70, 80 Light scattered particle measuring apparatus
2, 22, 32, 42, 62, 72, 82 Light source
3, 23, 43, 63, 73, 83 Incidence optical system
4, 24, 34, 44, 54, 64 Sample cell
5, 25, 35, 45, 75, 85 Emitting optical system
6, 26, 36, 46, 76, 86 Light detecting element
7 Amplifying circuit
8 Filter for noise removal
9 Arithmetic unit
10 Display unit
11 Stirring bar
12 Stirrer
27, 77, 87 Signal processing unit
28 External air temperature sensor
29, 39, 49, 59, 69 Heater
90, 92, 95, 96, 97 Cuvette
90a, 92a, 95a, 95b, 96a, 97a Well

1. An apparatus for measuring a physiologically active substance of biological origin, the apparatus comprising:
a sample cell for retaining a mixture liquid comprising a sample containing a physiologically active substance of biological origin like endotoxin and β-D-glucan and a reagent for inducing gelation with the physiologically active substance of biological origin;
light emitting portion for illuminating light beam from a light source to the mixture liquid in the sample cell;
convection generating portion for moving gel particles that are produced in the mixture liquid by partially heating/cooling the sample cell or the mixture liquid in itself and generating thermal convection in the mixture liquid in the sample cell;
light detecting portion for detecting scattered light which is incident light beam scattered by gel particles that are formed in the mixture liquid in the sample cell, and
measuring portion for measuring time series change in the number of gel particles based on the intensity of scattered light detected by the light detecting portion.

2. The apparatus for measuring a physiologically active substance of biological origin according to claim 1,
wherein the convection generating portion includes the portion for heating/cooling from the bottom part of the sample cell and/or the portion for heating/cooling from the top part of the sample cell.

3. The apparatus for measuring a physiologically active substance of biological origin according to claim 1,
wherein the convection generating portion includes a heater, and
the heater is in contact with the sample cell and supplies, via a thermistor, heat to a member having a hole at a site through which the light beam from a light source or scattered light passes.

4. The apparatus for measuring a physiologically active substance of biological origin according to claim 1,
wherein the convection generating portion includes a heater, and
the heater is an ITO heater with a light transmitting property.

5. The apparatus for measuring a physiologically active substance of biological origin according to claim 1, further comprising:
temperature measuring portion for measuring the temperature of the mixture liquid.

6. The apparatus for measuring a physiologically active substance of biological origin according to claim 5, further comprising:
external air temperature measuring portion for measuring the temperature of external air.

7. A method for measuring a physiologically active substance of biological origin by using the apparatus for measuring a physiologically active substance of biological origin described in claim 1, in which the time point at which the difference value of the number of gel particles produced in the mixture liquid in the sample cell per unit time is greater than the threshold value is taken as gelation detection time.

8. A method for measuring a physiologically active substance of biological origin by using the apparatus for measuring a physiologically active substance of biological origin described in claim 6 in which the time point at which time the difference value of the number of gel particles produced in the mixture liquid in the sample cell per unit time is greater than the threshold value is taken as gelation detection time,
wherein, by calculating temperature difference between the temperature of the mixture liquid and the temperature of external air based on output of the temperature measuring portion and the external air temperature measuring portion, velocity of thermal convection occurring in the mixture liquid is calculated and the threshold value is adjusted in accordance with the velocity of thermal convection.

9. A method for measuring a physiologically active substance of biological origin by using the apparatus for measuring a physiologically active substance of biological origin described in claim 6 in which the time point at which time the difference value of the number of gel particles produced in the mixture liquid in the sample cell per unit time is greater than the threshold value is taken as gelation detection time,
wherein, by maintaining the temperature difference between the external air temperature and the temperature of a site at which the sample cell is heated/cooled is kept at constant level based on output of the external air temperature measuring portion and output of the temperature measurement portion, velocity of thermal convection occurring in the liquid is kept constant irrespective of temperature of external air.

10. An apparatus for measuring a physiologically active substance of biological origin by producing a mixture liquid containing an AL reagent, which contains AL as amoebocyte lysate of limulus, and a sample, which contains predetermined physiologically active substance of biological origin, and detecting aggregation or gelation of proteins derived from a reaction between the AL and the physiologically active substance in the mixture liquid to detect the physiologically active substance contained in the sample or measure the concentration of the physiologically active substance, the apparatus comprising:
a sample cell for retaining the mixture liquid;
light emitting portion for illuminating light beam from a light source to the mixture liquid in a sample cell,
light detecting portion for detecting the light, which is illuminated from the light emitting portion, and scattered from the gel particles produced in the mixture liquid in the sample cell, and
measuring portion for measuring time series change in the number of gel particles based on the intensity of forward scattered light component which is scattered in direction of the optical axis of outgoing light opposite side of the sample cell from the optical axis of incident light illuminated from the light source to the sample cell.

11. The apparatus for measuring a physiologically active substance of biological origin according to claim 10, further comprising:
convection generating portion for moving gel particles that are produced in the mixture liquid by partially heating/cooling the sample cell or the mixture liquid in itself and generating thermal convection in the mixture liquid in a sample cell.

12. The apparatus for measuring a physiologically active substance of biological origin according to claim 10,
wherein the measuring portion includes a measurement system for detecting output of the forward scattered light component, and
the measurement system includes:
a first lens system which collects, among the light scattered from the mixture liquid in a sample cell, forward scattered light component which is scattered in direction of the optical axis of outgoing light opposite side of the sample cell from the optical axis of incident light illuminated from the light source to the sample cell, and emits the collected light as parallel light,
a transparent plate having a dark spot formed thereon for blocking light components having the same axis as the optical axis of outgoing light included in the parallel light which passes through without being scattered in the mixture liquid,
a second lens system for collecting the parallel light not including the light components blocked by the dark spot,
a pin hole for allowing partial pass through of the light collected by the second lens system, and
forward scattered light detecting portion for detecting the light passed through the pin hole.

13. The apparatus for measuring a physiologically active substance of biological origin according to claim 10,
wherein the measuring portion includes a measurement system for detecting intensity of the forward scattered light component, and
wherein the measurement system includes:
a third lens system which collects, among the light scattered from the mixture liquid in a sample cell, the forward scattered light component which is scattered in direction of the optical axis of outgoing light opposite side of the sample cell from the optical axis of incident light illuminated from the light source to the sample cell,
a pin hole which has been formed outside the axis of the optical axis of outgoing light, for blocking the outgoing light collected by the third lens system and passed through the mixture liquid without being scattered by it, and also for allowing partial pass through of the forward scattered light component collected by the third lens system, and
forward scattered light detecting portion for detecting the light passed through the pin hole.

14. The apparatus for measuring a physiologically active substance of biological origin according to claim 13,
wherein, regarding the direction of the optical axis of outgoing light, the pin hole is formed on a site other than light collection point at which the outgoing light passed through the mixture liquid without being scattered is collected by the third lens system.

15. The apparatus for measuring a physiologically active substance of biological origin according to claim 10,
wherein the measuring portion includes a multichannel measurement system, and
the light emitting portion divides light from a single light source into light for the multichannel and the light is illuminated on the mixture liquid in a plurality of sample cells corresponding to each channel.

16. A cuvette used for the apparatus for measuring a physiologically active substance of biological origin described in claim 15, wherein the cuvette is formed by comprising heat resistant glass which can be sterilized by dry heat and comprising sample cells corresponding to each channel.

17. The cuvette according to claim 16, wherein the sample cells corresponding to each channel are formed to be in a single row.

18. The cuvette according to claim 16, wherein the sample cells corresponding to each channel are formed to be in two rows.

19. The cuvette according to claim 16, wherein blocking portion for preventing incorporation of scattered light from a mixture liquid in one sample cell to a neighboring sample cell is installed between the one sample cell and the neighboring sample cell.

20. The cuvette according to claim 16, comprising:
an outside cuvette which is formed of a non-light transmitting material and has a plurality of sample cells formed therein and a hole through which light can pass through or a window through which light can transmit is formed at a site which corresponds to center of the sample cell on the lateral surface and/or bottom surface of the cuvette, and
inside cuvettes which are formed of heat resistant glass, have external shapes almost the same as the internal shapes of the sample cells, and can be inserted into the sample cells.
